(19) United States
(12) Patent Application Publication
(10) Pub. No.: US 2024/0000982 A1
(43) Pub. Date: Jan. 4, 2024

(54) METHOD OF EXCITING A MECHANICAL RESONANCE IN A STRUCTURAL COMPONENT OF A MICROORGANSIM

(71) Applicant: MedScienceSwiss AG, Küsnacht (CH)

(72) Inventor: Peter Ernst VOLLENWEIDER, Küsnacht (CH)

(21) Appl. No.: 18/265,084

(22) PCT Filed: Dec. 1, 2021
(86) PCT No.: PCT/EP2021/083802

§ 371 Date: Jun. 2, 2023

(30) Foreign Application Priority Data
Dec. 3, 2020 (WO) PCT/EP2020/084421
Apr. 30, 2021 (CH) 00472/21

(51) Int. Cl. A61L 2/02

(52) U.S. Cl. CPC A61L 2/02

(57) ABSTRACT

Method of exciting an electro-magnetic mechanical resonance in a structural component (2, 3) of a microorganism (1), the method comprising exposing said microorganism to an oscillating magnetic field (H), which oscillates at least at a first frequency, characterized in that said first frequency corresponds to a frequency of a mechanical resonance of said structural component. The invention is further directed to a method of selecting effective operating parameters to perform the invention. Under further aspects, the invention relates to applications of the method in in the technical fields of water treatment, nutrition industry, cell culture industry or paper industry as well as in the general reduction or limitation of the reproduction of specific germs in all areas, as well as in human and animal tissue cultures, ex vivo (extracorporal) of blood preparations. In a specific embodiment of the invention, the microorganism is brought in contact with magnetic nanoparticles, which are designed to attach themselves to the structural component of the microorganism.

The invention addressed herein relates to a method of exciting a mechanical resonance in a structural component of a microorganism and to a method of selecting effective operating parameters to perform the invention. Under further aspects, the invention relates to applications of the method in various technical fields and to a coil arrangement for performing the method.

In various technical fields, activity of microorganisms needs to be reduced or avoided. A well-known approach is to use chemicals, in particular antibiotics, to achieve this goal. This approach has numerous undesired side-effects, such as polluting the environment with toxic substances or creating multi-resistant germs.

The object of the present invention is to provide an alternative method for reducing the activity of a microorganism. In particular, it is a goal to achieve this effect based on physical means.

This object is achieved by a method according to claim 1.

The method according to the invention is a method of exciting a mechanical resonance in a structural component of a microorganism. The method comprises exposing said microorganism to an oscillating magnetic field, which oscillates at least at a first frequency. The first frequency corresponds to a frequency of a mechanical resonance of said structural component.

Within the present patent application, the term microorganism is meant to include single-celled organisms, such as bacteria and archaea, as well as viruses. A structural component may e.g. be a bacterial membrane, a cell organelle of a bacterium or a virus capsid. A cell organelle is e.g. a flagellum or a mitochondrion or the germs membrane itself.

The idea of the inventor is to excite a resonance frequency, which may be estimated from observed movements of the specific microorganism. This resonance may be excited to a degree that leads to a damage of the respective structural component. In consequence, the activity of the microorganism may be reduced, or the microorganism may be destroyed.

The waveform of the oscillating magnetic field may be purely sinusoidal, i.e. being defined by a single frequency being said first frequency. The waveform may be more complex, e.g. involving higher order harmonics of the first frequency. The waveform may e.g. have a saw tooth form or the form of rectangular pulses with a repetition frequency according to said first frequency. Various kinds of modulation are possible.

Variants of the method result from the features defined in claims 2 to 9.

In one embodiment of the method according to the invention, the first frequency is in the range up to 30 Megahertz, in particular in the range 0.01 Hertz to 400 KHz.

As an example, the applied magnetic field may oscillate at a frequency adapted to the rotational movements of the flagellae of a specific type of bacteria.

In one embodiment of the method according to the invention, the oscillating magnetic field is generated by driving an alternating current of the first frequency through a coil arrangement comprising at least one coil. The dimension of the coil may be adapted to the specific situation, e.g. to the size of a container in which the microorganism is held when performing the method. Such a container may e.g. be a microscope slide, a petri dish or an artificial vessel. In context of various technical field, such a container may also be a tube filled with water, an ingredient for preparing a nutrition product or any medium susceptible to microbial activity. The coil may e.g. be a circular coil with several hundred windings.

In one variant of the method using a coil arrangement, the coil arrangement comprises a pair of coils. The coils of the pair of coils are arranged on a common axis and spaced apart in direction of the axis. The microorganism is placed in a space between said coils of the pair of coils. Using pair of coils for generate the magnetic field has the advantage that access to the microorganism is possible from different sides in the space between the coils. The coil arrangement may form a so-called Helmholtz-coil.

In one variant of the method, the direction of said alternating current is either parallel in said coils of the pair of coils or is opposite in said coils of the pair of coils (gradient).

With parallel current direction, a region with a homogenous magnetic field can be created in the space between the coils and close to the axis. With current running in the first coils opposite to the direction of the second coil, a magnetic field gradient of the field component parallel to the axis is created. Homogenous field and field gradient may have different effects on different species of microorganisms. The relative current directions in the coils of the pair of coils is an operating parameter, which may be selected according to the needs.

In one embodiment of the method according to the invention, alternating current of said first frequency is driven through a first coil of said pair of coils and wherein alternating current of a second frequency is driven through a second coil of said pair of coils.

In this embodiment, separate current sources for the two coils are applied. Due to the difference between first and second frequency, there are periods of time during which the currents in both coils run in parallel, such that a homogenous field is created, and there are periods of time, during which a field gradient is created.

In one embodiment of the method according to the invention, a combination of duration of said exposing and of field strength of said oscillating magnetic field is selected such that a microbial activity is reduced, in particular such that said structural component of said microorganism is damaged.

The peptidoglycan architecture (contains N-Acetyl-glucosamin and N-Acetylmuroaminacetat) or/and the cytoplasma membrane may be destroyed by applying the method. The pressure inside bacteria is approx. 2 atmospheres. A little damage in the peptidoglycan membrane will lead to a blow-out.

In a variant of the method, which may be combined with any of the above-mentioned variants, the microorganism is brought in contact with magnetic nanoparticles at least while exposing the microorganism to the oscillating magnetic field.

Nanoparticles have a size between 1 and 100 nanometers in diameter. The nanoparticles may be brought in contact with the microorganism already before exposing the microorganism to the oscillating magnetic field. The magnetic nanoparticles may be permanently magnetized, they may e.g., comprise hard-magnetic material. As another example, the magnetic nanoparticles may be magnetized by a magnetic field, to which they are exposed, such as the oscillating magnetic field applied in the method according to the present invention. The magnetic nanoparticles may comprise iron or cobalt, in particular, the magnetic nanoparticle may comprise ferrite oxide or iron sulfate. The nanoparticles may comprise other magnetizing molecules.

The magnetic particles may consist only of substances, which have no poisonous effect to living cells. The magnetic particles may comprise a core of magnetic material and a coating, which coating need not to be magnetic and which prevents contact between the core and a surrounding, such as for example with a bodily fluid. For example, the magnetic particles may comprise ferromagnetic material or superparamagnetic material. The latter has the advantage that no magnetization of the particles is present once an external magnetic field is switched off and sticking together of the particles due to their magnetism is avoided.

In a specific embodiment of the above variant, the nanoparticles are designed to attach themselves to said structural component of said microorganism.

They may for example attach themselves to the outer surface of the cell membrane of the microorganism. They may be designed to be able to enter into the microorganism and then attach themselves to a cell organelle. Designing the nanoparticles to have this attaching property may involve applying a specific coating to the nanoparticles. The coating may be selected to attach to specific surfaces defined by their chemical properties.

An attachment to the surface of the bacterial membrane is conceivable. Magnetic nanoparticles inside the germs or on their surface could cause an amplified resonance and increase the damage potential to the membrane of the bacteria or other germs.

With this embodiment, the effect of the oscillating field onto the structural component is much stronger and the mechanical resonance of the structural component is more efficiently excited. Microbial activity is reduced more efficiently when this embodiment of the invention is applied.

The inventor has observed that the oscillating magnetic fields affect the germs also without using magnetic nanoparticles according to the above discussed embodiment.

On the one hand, the germinal membranes peptidoglycans are affected by the generation of a specific resonance. The peptidoglycan architecture (contains N-Acetyl-glucosamin and N-Acetylmuroaminacetat) or/and the cytoplasma membrane may be destroyed by applying the method. The pressure inside bacteria is approx. 2 atmospheres. A little damage in the peptidoglycan membrane will lead to a blow-out.

Static magnetic fields interact with moving charges via the Lorentz-force:

F=q[v×B],

where q is the charge and v the velocity-vector. Since the force on opposite charges acts in opposite directions, electric dipoles show the tendency to align orthogonal to the velocity. This might slow down the vital coherent motion.

Alternating magnetic fields induce rotating electrical fields according to Maxwell's first field equation:

rot E=−∂B/∂t.

In a conducting non-magnetic medium this rotating field induces eddy-currents. The strength of these eddy-currents is proportional to the conductivity of the electrolyte and to the frequency and strength of the magnetic field oscillations. These eddy currents may disturb the coherent intra-cellular flow, too. Furthermore, these effects may induce strong enough oscillations for the cellular wall to break open when a cellular resonance frequency is met.

Further in the scope of the invention lies a method according to claim 10.

It is a method of rating a first frequency regarding efficacity.

The method comprises the steps
observing a pre-treatment activity of a first microorganism,
exposing said first microorganism to an oscillating magnetic field oscillating at said first frequency,
observing a post-treatment activity of said first microorganism,
determining a rating of efficacity for said first frequency in dependence of the difference between said post-treatment activity and said pre-treatment activity.

The microorganisms of a pure bacterial culture may undergo the rating according to this method. To evaluate the effectiveness of the above-described frequency application, high tech fluorescence microscopy, quantitative smears, counting chambers and comparison cultures after incubation can be used. The rating of efficacity may be a killing rate specific for the first frequency. Identically prepared bacterial cultures may be used to determine and comparing the efficacity of different choices for the first frequency.

The rating of efficacity may in addition include the aspect, that another type of bacteria, virus or other cells undergoing the same treatment is not affected or damaged. For compatibility in the experimental field, human HEKs cells or lymphocytes can be seeded with the sample germs and thus the same application can be performed to kill or damage the germs. No damage of the inoculated cells in culture is shown by the electromagnetic therapy application described above.

In one embodiment of the method of rating a first frequency regarding efficacity, the rating of efficacity applies to a set of values of operating parameters of the method according to the invention. The set comprises at least the first frequency.

Together with rating the effect of the first frequency, other operating parameters may be selected appropriately to achieve a high efficiency.

In one embodiment of the method of rating a first frequency regarding efficacity, the set of values of operating parameters is defined as
the first frequency, and/or
the first frequency and the relative direction of the alternating current in a pair of coils, and/or
the first frequency and the second frequency.

The relative direction of the current may be selected to be parallel or opposite in a pair of coils, e.g. in a Helmholtz-pair as discussed in more detail below.

Further in the scope of the invention lies a method according to claim 13.

It is a method of determining a species-specific frequency of the method according to the invention. The method comprises repeatedly performing the method according to the invention with various values of said first frequency. In each repetition of the method of rating a first frequency regarding efficacity is applied to a microorganism of a first species. A table of ratings of efficacity in dependency of said first frequency is established, wherein the frequency with the highest rating of efficacity is selected as the species-specific frequency for said first species.

This method can be repeated for a second species, a third species and so on. A table or a database of species-specific frequencies may be established based on the method.

In one embodiment of the method, a frequency range for the first frequency is estimated based on observed movements of the first species of microorganisms. Various values of the first frequency are selected from the frequency range, which is estimated based on observed movements.

This embodiment of the method has the effect that a species-specific frequency may be found in a short time. Testing a huge list of possible frequencies for their effect on a certain species of microorganism can be avoided. A concentration of the tests to an interesting region of frequencies is possible. As an example, species-specific flagella movements may be systematically observed and screened for characteristic frequencies. These results may be collected in a database, which in turn is the basis for setting up tests for identifying the most effective frequency of an oscillating magnetic field.

The invention is further directed to a use of the method according to any one of claims 1 to 9 for reduction of microbial activity in the technical fields of water treatment, nutrition industry, cell culture industry or paper industry as well as in the general reduction or limitation of the reproduction of specific germs in all areas, as well as in human and animal tissue cultures, external treatment of blood preparations.

The invention is further directed to a coil arrangement according to claim 16. This coil arrangement is adapted for performing the method according to the invention. The coil arrangement is formed of a number of mutually isolated loops of flexible wire, in particular of stranded wire. The loops surround a free space into which said microorganism can be placed. E.g., the free space has a diameter large enough to receive a container containing said microorganism when performing the method according to the invention. This container may be a petri dish, as an example. It may as well be a part of a human or animal body infected by said microorganism. The loops are connected in series through a multiple connector pair, allowing for connecting and disconnecting several of said loops simultaneously.

The multiple connector may be a DSUB-15 connector pair consisting of a male and a female connector, having 15 corresponding connector pins or connector sockets, respectively. As an example, an inlet wire and 15 wires connected to pins of the male part of the multiple connector may be grouped in one tube. The wires may be connected to transposed positions of the sockets of the female part, wherein the inlet wire is connected to the first socket, wherein the first pin is connected to the second socket, and so on, such that last pin (pin 15 in this example) ends as outlet wire on the side of the tube where the female part of the connector is attached. The individual wires inside the tube are mutually isolated from each other. The whole tube may be arranged as one circular loop, which in this case results in a coil arrangement having 16 loops. The whole tube may be arranged in multiple windings, too, such that each winding of the tube produces 16 loops of the coil arrangement. E.g. 16 windings of the tube result in a coil arrangement having 256 loops in total. This coil arrangement allows arranging a large number of loops in a flexible and efficient way. The flexibility of the coils in the arrangement makes the whole arrangement suitable for local application of magnetic fields.

Such a coil arrangement may e.g. be formed by approximately circular loops, having an average diameter of 90 mm and surrounding a cylindrical free space of diameter 77-80 mm. The wires may consist of copper strands having a total cross section of 0.3 mm². In this case, with 256 loops and 16 windings of the tube, a tube length of approximately 4.5 meters results, which length can be handled relatively easy while producing the windings of the tube. By connecting the male and female parts of the multiple connector, the full number of loops are connected in series in one step.

The coil arrangement may be designed to produce in the center of the coil arrangement a magnetic field in the milli Tesla (mT) range, when a current of 1-4 Ampère (A) flows through the coil arrangement. The coil arrangement may be connected to a power supply in a way that a capacitor is connected in series to the coil. To be select an appropriate size for the capacitor, a capacitor decade consisting of several capacitors connected in parallel to each other may be used, wherein the individual capacitors may be activated by closing a switch in the respective one of parallel branches. E.g. the combination of 100 pico Farad (pF), 1 nano Farad (nF), 10 nF, 100 nF and 1 micro Farad (mF) covers a range suitable for the coil dimensions as discussed in the above example.

According to a further example, the coil arrangement has a larger average diameter of 200 mm and has the double number of windings of the tube, compared to the above embodiment. This further embodiment has 32 windings of the tube and thus comprises 512 coil loops in total. This leads to a tube length of approximately 10 meters.

Under the rough assumption, that only half of the magnetic field energy is situated within the coil, an approximation of the magnetic field strength achievable with a coil arrangement fed with current I can be calculated according to the following formula:

B(t)≈\sqrt{μ₀L/2V}*I(t)

where β₀ is the induction constant and V the mean volume of the coil, B(t) the time dependent magnetic field and I(t) the time dependent current in Ampere. Example:
L=20 mH (milli Henry), V=0.001 m³ (cubic meters)→B(t)≈3.5 mT/A*I(t).

The invention shall now be further exemplified with the help of figures. The figures show:

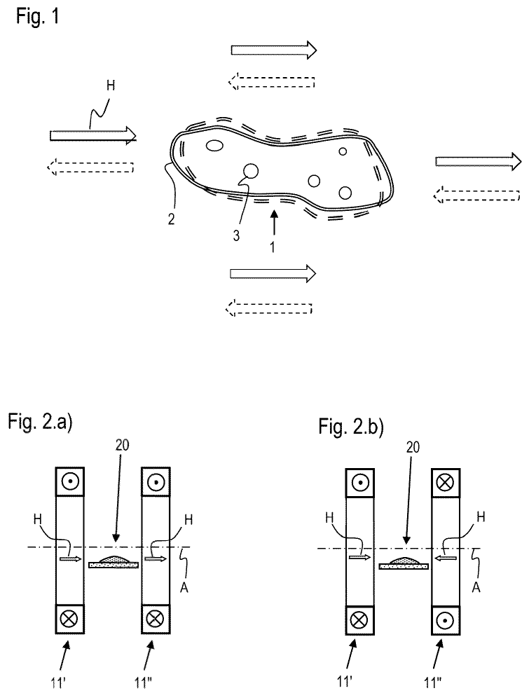

FIG. 1 a schematic view of a situation occurring during the method according to the invention;

FIG. 2.a) and 2.b) cross-sections through coil-arrangements used for performing embodiments of the method;

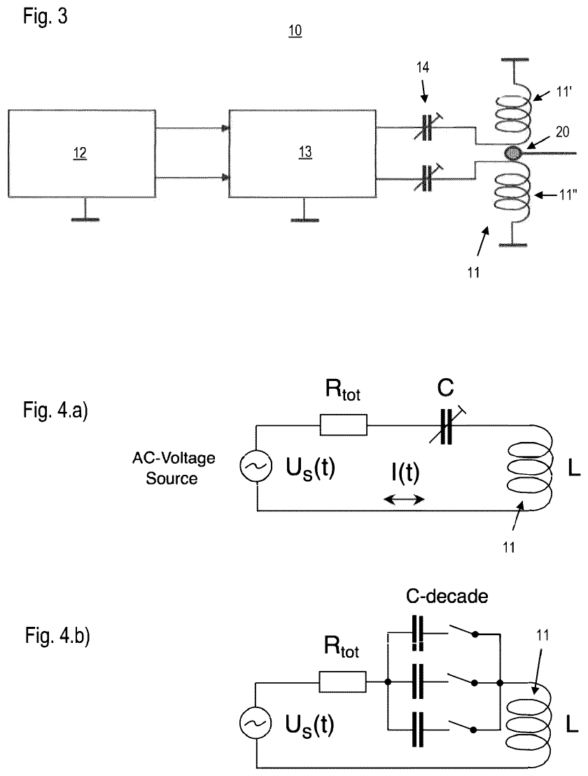

FIG. 3 a schematic view of an apparatus for performing the method according to the invention;

FIG. 4.a) and 4.b) schematic views of variants to drive an alternating current through the coil arrangement;

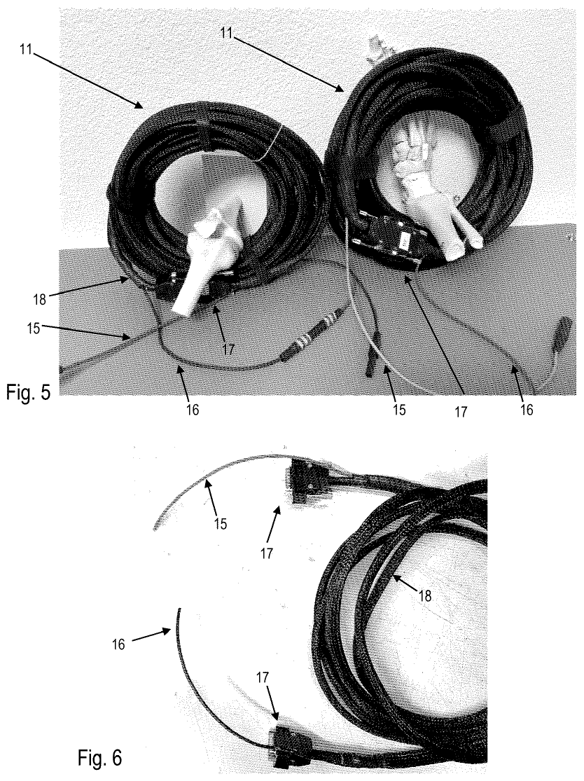

FIG. 5 a photography of embodiments of flexible coil arrangements;

FIG. 6 a photography of an embodiment of a coil arrangement with male and female connector parts in the disconnected state;

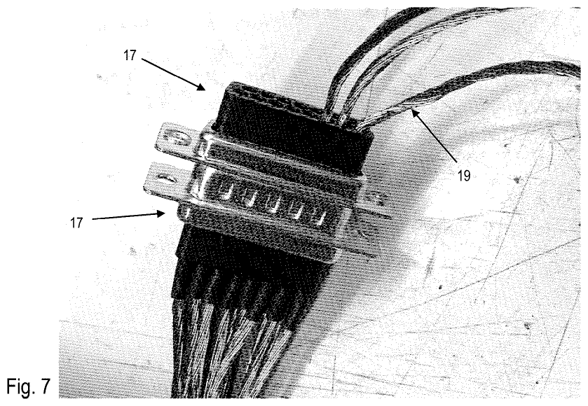

FIG. 7 a photography of a detail of partially assembled coil arrangement.

FIG. 1 shows schematically and by means of an illustrative simplified example, a situation occurring during the method according to the invention. A microorganism 1 is exposed to an oscillating magnetic field H. The orientation and oscillating polarity of the magnetic field is symbolically indicated by arrows. The microorganism shown has a cell membrane 2 and organelles 3. In the case shown, the cell membrane is the structural component, which undergoes a periodical mechanical deformation. Extreme positions of the deformation are shown in double lines and in double dashed lines, respectively. The orientation of the magnetic field after half a period of one oscillation of the magnetic field is shown as arrows with dashed lines. The frequency of the applied oscillating magnetic field corresponds to the resonance frequency related to the periodical mechanical deformation.

FIG. 2.a) shows a cross section through coil-arrangement comprising a pair of coils arranged as Helmholtz-pair. Both coils of the pair of coils are arranged on a common axis A, which is indicated as dash-dotted line. First coil 11′ and second coil 11″ of the pair of coils. An oscillating current is driven through both the coils such that the current runs in parallel in both coils. This way, the magnetic field H of the first coil is oriented in the same direction as the field of the second coil. The magnetic fields produced by the coils add up to a homogeneous magnetic field in a region between the coils and close to the axis. A container 20, which carries microorganisms, is placed in the space between the coils.

FIG. 2.b) shows a cross section through coil-arrangement similar to the one shown in FIG. 2.a), but with the difference, that the current is driven through the coils of the pair of coils in opposite direction. This way, the magnetic field generated by the first coil is directed in opposite direction compared to the field direction of the second coil. The axial component of the magnetic field, i.e. the field component parallel to the axis A, has a gradient form in the space between the two coils of the pair of coils. The microorganism placed in the space between the coils are exposed to an oscillating magnetic field gradient.

Coil-arrangements as shown in FIG. 2.a) and FIG. 2.b) may be driven by a single current source, when the two coils are connected in series. Alternatively, each coil may be driven by a separate current source. In the latter case, the frequency of the oscillation may be selected differently for the current in the first and the second coil. In this case, the magnetic field created oscillates between the situations shown in FIG. 2.a) and FIG. 2.b) as extremal situation.

FIG. 3 shows a schematic view of an apparatus 10 for performing the method according to the invention. The apparatus comprises a coil-arrangement 11, which comprises a first coil 11′ and a second coil 11″. The geometry of the coil-arrangement may e.g. be a Helmholtz-pair, as shown in FIG. 2.a) or FIG. 2.b). A two-channel RF-frequency generator 12 has two output channels, which deliver each an oscillating signal oscillating at a selectable first frequency and second frequency, respectively. A two-channel broad band power amplifier amplifies the signals of the RF-frequency generator such that the first coil 11′ and the second coil 11′ can be driven with an oscillating current of the first and the second frequency. A trimmer 14, i.e. an adjustable capacity, for phase compensation is connected in series to each of the coil. The adjustable capacity may be built as a capacitance decade dimensioned for high voltages across the coils, e.g. for voltages in the range 50 V to 5 KV.

FIG. 4.a) shows a variant of driving an alternating current I(t) through the coil arrangement 11, symbolically indicated as coil having inductance L. The coil arrangement may have more complicated structure than indicated by the symbol in this schematic diagram. A variable capacitor C is connected in series to the coil arrangement. The complete configuration has a total resistance Rₜₒₜ, which includes the DC resistance of the coil, a resistance due to the frequency dependent skin effect, the dielectric loss of the capacitor and the output impedance of the source. Applying an oscillating voltage Uₛ(t), provided by an AC-voltage source, leads to a current I(t) flowing through the coil arrangement and producing the oscillating magnetic field used for the method according to the invention. The voltage source may be able to provide a DC voltage, too, in particular to provide a DC-offset in addition to the AC-voltage. The capacitor may be set to a capacitance value, which fits to the inductance of the coil arrangement. An alternative to the variable capacitor is a capacitor decade, as shown in FIG. 4.b).

FIG. 5 shows a photography of two embodiments of flexible coil arrangements. They are positioned around models of parts of a human skeleton, in order to illustrate the possibility of locally applying oscillating magnetic fields by means of these coil arrangements. The coil arrangements shown comprise multiple wires in a common tube and connected at both ends of the tube by a multiple connector pair, in the case shown by DSUB-15 connectors. Inlet and outlet wire are provided with connectors that enable connecting the coil arrangement to a power source. The tube is wound to several circular loops. In the example coil arrangement shown on the left half of the photo, the tube forms 16 loops. The tube, which in this example is formed as a braided sleeve, contains 16 wires, such that a total of 256 windings results in the complete coil arrangement.

FIG. 6 shows a photography of an embodiment of a coil arrangement with male and female connector parts in the disconnected state. In this state, it is simple to position the coil arrangement at a new place, where magnetic fields are to be applied. Before starting operation, the male and female connector parts are connected, and the inlet and outlet wire are connected to a power supply.

FIG. 7 shows a photography of a detail of partially assembled coil arrangement. Stranded wires are connected to the connector part shown in the lower part of the photo. In the upper part, only three positions of the DSUB-15 connector are connected in the state of assembling as shown here.

### LIST OF REFERENCE SIGNS

1 microorganism
2 cell membrane
3 organelle
10 apparatus for performing the method
11 coil arrangement
11′ first coil (of pair of coils)
11″ second coil (of pair of coils)
12 two channel RF-frequency generator
13 two channel broad band power amplifier
14 trimmer for phase compensation
15 inlet wire
16 outlet wire
17 multiple connector
18 tube
19 stranded wire
20 sample/container for microorganism
A axis
H magnetic field

1. Method of exciting a mechanical resonance in a structural component (2, 3) of a microorganism (1), the method comprising exposing said microorganism to an oscillating magnetic field (H), which oscillates at least at a first frequency,
characterized in that
said first frequency corresponds to a frequency of a mechanical resonance of said structural component.

2. Method according to claim 1, wherein said first frequency is in the range up to Megahertz, in particular in the range 0.01 Hertz to 400 kHz.

3. Method according to claim 1, wherein said oscillating magnetic field (H) is generated by driving an alternating current of said first frequency through a coil arrangement (11) comprising at least one coil (11′, 11″).

4. Method according to claim 3, wherein said coil arrangement (11) comprises a pair of coils, wherein the coils of the pair of coils are arranged on a common axis (A) and spaced apart in direction of said axis, and wherein said microorganism (1) is placed in a space between said coils (11′, 11″) of the pair of coils.

5. Method according to claim 4, wherein the direction of said alternating current is either parallel in said coils (11′, 11″) of the pair of coils or is opposite in said coils of the pair of coils.

6. Method according to claim 4, wherein alternating current of said first frequency is driven through a first coil (11′) of said pair of coils and wherein alternating current of a second frequency is driven through a second coil (11″) of said pair of coils.

7. Method according to claim 1, wherein a combination of duration of said exposing and of field strength of said oscillating magnetic field is selected such that a microbial activity is reduced, in particular such that said structural component of said microorganism is damaged.

8. Method according to claim 1, wherein said microorganism (1) is brought in contact with magnetic nanoparticles at least while exposing said microorganism to said oscillating magnetic field (H).

9. Method according to claim 8, wherein said magnetic nanoparticles are designed to attach themselves to said structural component of said microorganism.

10. Method of rating a first frequency regarding efficacity,
wherein the method comprises the steps
observing a pre-treatment activity of a first microorganism,
exposing said first microorganism to an oscillating magnetic field oscillating at said first frequency,
observing a post-treatment activity of said first microorganism,
determining a rating of efficacity for said first frequency in dependence of the difference between said post-treatment activity and said pre-treatment activity.

11. The method according to claim 10, wherein the rating of efficacity applies to a set of values of operating parameters of the method according to claim 1, the set comprising at least said first frequency.

12. Method according to claim 11, wherein said set of values of operating parameters is defined as
said first frequency, and/or
said first frequency and the relative direction of the alternating current in a pair of coils, and/or
said first frequency and said second frequency.

13. Method of determining a species-specific frequency of the method according to claim 1,
wherein the method comprises repeatedly performing the method according to one of claims 1 to 9 with various values of said first frequency,
wherein in each repetition of the method according to claim 10 is applied to a microorganism of a first species,
wherein a table of ratings of efficacity in dependency of said first frequency is established,
wherein the frequency with the highest rating of efficacity is selected as the species-specific frequency for said first species.

14. Method according to claim 13, wherein a frequency range for said first frequency is estimated based on observed movements of said first species of microorganisms and wherein said various values of said first frequency are selected from said frequency range.

15. Use of the method according to claim 1 for reduction of microbial activity in the technical fields of water treatment, nutrition industry, cell culture industry or paper industry as well as in the general reduction or limitation of the reproduction of specific germs in all areas, as well as in human and animal tissue cultures, ex vivo, i.e. extracorporal, treatment of blood preparations.

16. Coil arrangement (11) for performing the method according to claim 3, wherein the coil arrangement is formed of a number of mutually isolated loops of flexible wire, in particular of stranded wire, wherein said loops surround a free space into which said microorganism can be placed, wherein said loops are connected in series through a multiple connector pair, allowing for connecting and disconnecting several of said loops simultaneously.
